(19) United States
(12) Patent Application Publication
(10) Pub. No.: US 2013/0005786 A1
(43) Pub. Date: Jan. 3, 2013

(54) THERAPEUTIC INVENTION

(76) Inventors: Raju KAPOOR, London (GB); Gavin GIOVANNONI, London (GB)

(21) Appl. No.: 13/539,042

(22) Filed: Jun. 29, 2012

Related U.S. Application Data
(60) Provisional application No. 61/502,537, filed on Jun. 29, 2011.

(51) Int. Cl. A61K 31/4166; A61P 25/00

(52) U.S. Cl. 514/391

(57) ABSTRACT

The invention provides a method of treating acute demyelinating optic neuritis in a patient suffering therefrom, which method comprises administering a compound to the patient, wherein the compound is phenytoin or an analog or prodrug thereof. The compound acts as a neuroprotectant by partial blocking of voltage-gated sodium channels in axonal other cell membranes thereby reducing the entry of sodium into the respective cell in vivo. The treatment is particularly concerned with preserving vision after onset of the acute demyelinating optic neuritis.

### REFERENCE TO RELATED APPLICATIONS

This application claims priority to U.S. provisional patent application No. 61/502,537, filed Jun. 29, 2011.

### TECHNICAL FIELD

The present invention relates generally to methods and materials for the treatment of demyelinating optic neuritis to improve recovery.

### BACKGROUND ART

Demyelinating optic neuritis is the most common cause of acute and reversible visual failure in young adults of Northern European origin. Loss of vision usually develops over days and recovers over several weeks, but 10-15% of patients experience poor recovery of vision, or else slow recovery over several months²⁻⁴.

The acute inflammatory lesion in the optic nerve resembles the plaques found elsewhere in the central nervous system in multiple sclerosis (MS), and indeed optic neuritis is a manifestation of MS in some cases. However demyelinating optic neuritis is a distinct indication from MS, and occurs at a frequency of around 1-5/100,000/yr in the UK, and similar levels in the US and Australasia.

Present treatment using corticosteroids has little or no impact on the extent to which vision finally recovers after an attack of optic neuritis⁵⁻¹². Trials of intramuscular adrenocorticotropic hormone (ACTH) or oral prednisone, have shown no short or long term benefits, while others have indicated that intramuscular ACTH, intravenous methylprednisolone or retrobulbar triamcinolone can lead to faster visual recovery without influencing the final extent of the recovery. Thus, the North American Optic Neuritis Treatment Trial found that there was a small improvement in contrast sensitivity, visual field and color vision in patients treated with intravenous methylprednisolone followed by oral prednisone when studied at six months, but that these benefits were no longer apparent after one year.

Therefore, in the absence of an effective acute treatment, prevention or mitigation of residual disability from optic neuritis represents a major unmet need.

It has previously been thought that visual loss in optic neuritis occurred primarily because axons which become demyelinated, and which are exposed to inflammation¹³⁻¹⁵ cannot conduct action potentials reliably. Whereas conduction block due to demyelination and inflammation tends to be reversible over time, axonal degeneration is now considered to be the major cause of persistent residual disability¹⁶⁻¹⁸.

In particular, imaging of the retinal nerve fiber layer (RNFL) using optical coherence tomography (OCT), and of the optic nerve using magnetic resonance imaging, both demonstrate that acute optic neuritis is associated with significant volume loss, and that this correlates with impaired visual function¹⁹. Thus among the mechanisms of axonal injury, it appears that inflammation can interact with adaptive changes in the demyelinated axonal membrane, particularly an increased density of sodium channels, to favor an increased intra-axonal concentration of sodium ions²⁰, and thereby of calcium ions via the sodium-calcium exchanger^{20,21}.

Irrespective of the precise etiology or mechanism of the disease, it can be seen that the provision of novel treatments for acute optic neuritis would provide a contribution to the art.

Some of the present disclosure was presented on by one or more of the present inventors at a meeting on 30 Jun. 2010 in London, UK.

Some of the present disclosure was presented on by one or more of the present inventors at a meeting in January 2011 in New York, USA.

### DISCLOSURE OF THE INVENTION

The present invention concerns the treatment of acute demyelinating optic neuritis with voltage-gated sodium channel blockers, such as phenytoin or analogs thereof, to achieve neuroprotection and improve preservation of vision thereafter.

Partial blockade of voltage-gated sodium channels using drugs including phenytoin have previously been shown to be neuroprotective in several experimental models of inflammatory axonal injury²²⁻²⁶. Although the focus of this research has been on sodium channels occurring in the axonal membrane, it has separately been suggested that similar channels are found in cells of the immune system²⁷.

The present inventors now propose that neuroprotection can be achieved in acute optic neuritis by using the methods and materials described herein to achieve partial blocking of voltage-gated sodium channels in axonal and\or immune cell membranes.

In various aspects the present invention provides methods of treating acute (demyelinating) optic neuritis in a patient suffering therefrom, which method comprises administering a compound of the invention to the patient. Compounds of the invention are phenytoin or analogues or prodrugs thereof as described herein below.

Phenytoin has previously received marketing authorization for other indications, and in particular epilepsy.

Keltner et al (1972) TR. AMER. OPHTHAL. SOC., VOL. LXX, 1972 113-130 relates to the use of phenytoin in diseases of the optic nerve, particularly those caused by blood vessel blockage (“ischemic optic neuritis”) generally with a review of reversing existing damage. Such diseases are clinically and etiologically distinct from diseases of the nerve due to demyelination.

Ellenberger et al (1974) ARCH. OPHTHALMOL. 91: 435- from the same group also concerns ischemic disease. In the trial discussed neither visual acuity nor visual fields improved during treatment.

More recently Hains and Waxman (Investigative Ophthalmology & Visual Science, November 2005, Vol. 46, No. 11 2995, pp 4164-4169) reported the use of phenytoin in an experimental model of glaucoma. Again glaucoma is clinically and etiologically distinct from acute demyelinating optic neuritis. Waxman has also reviewed the use sodium channel blockers as neuroprotectants in multiple sclerosis (2008, NATURE CLINICAL PRACTICE NEUROLOGY 159-169).

Phenytoin has been used to treat paroxysmal phenomena (such as trigeminal neuralgia) and epilepsy, in people with MS, where the safety profile of maintenance treatment appears to be essentially the same as in other disorders where its use is indicated³⁴. Trigeminal neuralgia occurs in approximately 1-2% of patients suffering from multiple sclerosis³⁴. Its use in treating trigeminal neuralgia or other painful sensations (dysaesthesia) is generally reserved for where other approaches have not worked. It should be appreciated that the phenytoin in this context was a purely symptomatic treatment, and did not suggest disease modification or prevention of disability. These disclosures did not teach its use in the present indication, patient groups, or using the treatment regime described herein, or for the same purposes as described herein.

Phenytoin has also been suggested for use in treating Experimental Autoimmune Encephalomyelitis (EAE)²³ as well as models of ischemia or hypoxia^{35,36} and neural trauma³⁷.

In the present invention the treatment is intended to reduce residual visual dysfunction resulting from said acute demyelinating optic neuritis. Furthermore in the present invention the treatment is intended to regulate (e.g. to reduce or inhibit) axonal degeneration associated with said acute demyelinating optic neuritis. None of these previous publications teach of suggest the present invention.

One aspect of the invention pertains to a pharmaceutical composition comprising a compound as described herein and a pharmaceutically acceptable carrier or diluent, for use in the methods described herein concerning acute demyelinating optic neuritis.

One aspect of the present invention pertains to a compound as described herein for use in a method of treatment of acute demyelinating optic neuritis by therapy.

One aspect of the present invention pertains to use of a compound as described herein, in the manufacture of a medicament for use in the treatment or prophylaxis of acute demyelinating optic neuritis as described herein.

Some aspects and embodiments of the present invention will now be discussed in more detail:

### Compounds of the Invention

Compounds of the present invention generally include phenytoin (5,5-diphenylhydantoin) and analogs or prodrugs thereof.

For example, compounds of the present invention are selected from compounds of formula (I):

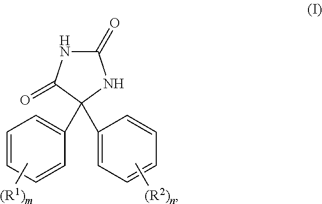

and their salts, solvates, hydrates, prodrugs and isomers thereof,
wherein:
each R¹ and each R² are independently selected from the group consisting of C₁₋₄alkyl, —OR^{O1}, —NR^{N1}R^{N2}, —X, and —CX₃;
each R^{O1}, each R^{N1} and each R^{N2} are independently selected from the group consisting of hydrogen and C₁₋₄alkyl;
each X is independently selected from F, Cl, Br and I;
m is selected from 0, 1, 2 and 3; and
n is selected from 0, 1, 2 and 3.

A preferred compound is phenytoin (5,5-diphenylhydantoin).

In another embodiment the compound is an analog of phenytoin.

In another embodiment the compound is a pro-drug of phenytoin.

Compounds which may be used in the present invention include fosphenytoin, hydroxyphenytoin, 5-(3-hydroxyphenyl)-5-phenylhydantoin, 5-phenyl-5-(4-hydroxyphenyl)hydantoin glucuronide, ropitoin, ropitoin hydrochloride, 5-(2-hydroxyphenyl)-5-phenylhydantoin, 5-(3,4-dihydroxy-1,5-cyclohexadien-1-yl)-5-phenylhydantoin, N-aminodiphenylhydantoin, 5-(3,4-dihydroxyphenyl)-5-phenylhydantoin, PC-796, 5-p-methylphenyl-5-phenylhydantoin, 1-acetyl-3-acetoxy-5,5-diphenylhydantoin, 3-hydroxymethylphenytoin N,N-dimethylglycine ester, 3-(hydroxymethyl)phenytoin N,N-dimethylaminoethyl carbonate, 5-(4-hydroxy-3-methoxyphenyl)-5-phenylhydantoin, 3-pentanoyl-5,5-diphenylhydantoin, 3-(2-propylpentanoyl)-5,5-diphenylhydantoin, 5,5-bis(4-hydroxyphenyl)hydantoin, 3-(hydroxymethyl)phenytoin, phenytoin dihydrodiol, 4-aminophenytoin, N,N-dichlorophenytoin, diphenylthiohydantoin, diphenylhydantoin-3-phenyltricarbonylchromium ethyl acetate, 5,5-diphenylhydantoin-3-valerate-bovine serum albumin, phenytoin-1-methylnicotininate, 2-cyanoguanidinophenytoin, phenytoin-bis-hydroxyisobutyrate, N-acetylphenytoin, diphenylhydantoic acid, N′-3-oxymethylglucuronide phenytoin, diphenylhydantil, 5-(4′-fluorophenyl)-5-phenylhydantoin.

Other compounds include Dantrolene, Azumolene,

Other compounds include 5,5-bis(4-trifluoromethylphenyl)hydantoin, 5,5-bis(4-methylphenyl)hydantoin, 5,5-bis(4-methoxyphenyl)hydantoin, 5-(4-methoxyphenyl)-5-phenylhydantoin, and 5-(4-dimethylaminophenyl)-5-phenylhydantoin (see Mahmoodi et. al, Russ. J. Org. Chem., 40(3), 2004, 377-382).

Other preferred compounds of the invention are discussed hereinafter.

Compounds of the invention will share the biological activity of phenytoin i.e. will be capable of blocking, or partially blocking, voltage-gated sodium channels, thereby reducing the entry of sodium into the cell in vivo.

Thus it will be appreciated by those skilled in the art that where “phenytoin” is described herein, each and every part of the disclosure applies mutatis mutandis to other compounds of the invention as described herein, which are phenytoin analogs or prodrugs. In each case, the treatment using the compound causes partial blockade of voltage-gated sodium channels in the context of, and for the purposes of, the treatments described herein.

### Dosage Regimen

In one aspect of the present invention pertains to a method of treatment of acute demyelinating optic neuritis in the subject, the method comprising administering to said subject a prophylactically or therapeutically effective amount of a compound as described herein, preferably in the form of a pharmaceutical composition.

Previous experience has demonstrated that therapeutically relevant serum concentrations of phenytoin can be achieved quickly when phenytoin is taken orally in an acute clinical setting.

In the methods of the invention, the dose regimen of the compounds is preferably such as to achieve a therapeutic plasma concentration of between about 5 to 30 mg/ml, more preferably between about 10-20 mg/ml. This range is intended to block sodium channels; phenytoin can also block calcium and potassium channels^{38,39}, but only at higher concentrations.

Administration can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell(s) being treated, and the subject being treated. Single or multiple administrations can be carried out with the dose level and pattern being selected by the treating physician or clinician.

In general, a suitable dose of the compound is in the range of about 1 mg to about 25 mg per kilogram body weight of the subject per day e.g. 2, 3, 4, 5, 6, 7, 8, 9, 10, 11, 12, 13, 14, 15, 16, 17, 18, 19, 20, 21, 22, 23, 24 mg.

The dosing may be split into loading and maintenance doses.

Optionally a loading dose is between and 5 and 25, e.g. 10 and 20 mg/kg/day.

Optionally a maintenance dose is between and 1 and 10 mg/kg/day.

Preferably a maximum daily maintenance dose is less than 500, 400 or 300 mg/day.

Thus in one embodiment, to achieve a therapeutic concentration, phenytoin is loaded using a total dose of 15 mg/kg (which may be rounded up to the nearest 100 mg) divided into three equal doses given once daily on 3 consecutive days to achieve a therapeutic drug concentration gradually over the 3 days.

This will be followed by a daily maintenance dose of 4 mg/kg once daily (which may be rounded up to the nearest 50 mg, with a maximum of 300 mg).

In some embodiments, the compound is administered to a human patient according to the following dosage regime: about 50 mg, 3 times daily.

In some embodiments, the compound is administered to a human patient according to the following dosage regime: about 100 mg, 3 times daily.

In some embodiments, the compound is administered to a human patient according to the following dosage regime: about 150 mg, 2 times daily.

In some embodiments, the compound is administered to a human patient according to the following dosage regime: about 200 mg, 2 times daily.

### Patient Groups and Timing of Administration

Patients will preferably have a clinical diagnosis of acute unilateral optic neuritis.

In one embodiment the patient also has a diagnosis of MS.

In one embodiment the patient does not have a diagnosis of MS.

In one embodiment the patient may have a visual acuity prior to treatment of less than 6/9 (M), 20/30 (Ft), or above LogMAR 0.20.

In the methods of the invention, the compound is preferably administered to patients shortly after onset of the acute optic neuritis. This is intended to minimize axonal injury as driven both by increased expression of axonal sodium channels and by inflammation.

Preferably the treatment takes place within 1, 2, 3, 4, 5, 6, 7, 8, 9, 10, 11, 12, 13 and 14 days of onset or diagnosis of optic neuritis. Preferably the treatment is within 1, 2, 3, 4, 5, 6 or 7 days, preferably within 1,2 or 3 days of onset of optic neuritis.

Preferably the treatment is sustained until both inflammation and axonal membrane readaptation have subsided. It is preferred that the neuroprotection is applied such that the sodium channels are partially blocked throughout the inflammatory phase (which is believed to persist for an average of 4 weeks from the onset of optic neuritis^{13,42}.

Thus, in the methods of the invention, the compound is preferably administered to patients for about or at least 4, 5, 6, 7, 8, 9, 10, 11, 12, 13, 14, 15, 16, 17, 18, 19, 20 weeks, or about or at least 1, 2, 3, 4, 5, or 6 months from the onset of optic neuritis.

In embodiments the compound may be administered to patients for less than 4, 5, or 6 months from the onset of optic neuritis.

A preferred treatment is for about 14 weeks or 3 months.

### Combination Therapies

In embodiments the present invention may be combined with other therapies, whether symptomatic or disease modifying.

For example the therapeutic method described herein may be combined with treatment with an oral prednisone e.g. methylprednisolone, which by way of non-limiting example could be given orally (e.g. 500 mg daily for 5 days) or intravenously treatment^{57,58}.

Combination with intramuscular adrenocorticotropic hormone (ACTH) is also encompassed.

Combination treatment with corticosteroids and/or standard disease modifying treatment at the discretion of the treating physician is also encompassed by the present invention.

Further aspects of the invention as they relate to treatment, prophylaxis, dosage formulation, and assessment of efficacy are given hereinafter.

### Compounds of the Invention

As explained hereinbefore, the present invention relates to use of phenytoin or a phenytoin analog.

Accordingly, compounds of the present invention are selected from compounds of formula (I):

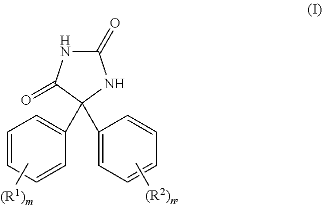

and their salts, solvates, hydrates, prodrugs and isomers thereof,
wherein:
each R¹ and each R² are independently selected from the group consisting of C₁₋₄alkyl, —OR^{O1}, —NR^{N1}R^{N2}, —X, and —CX₃;
each R^{O1}, each R^{N1} and each R^{N2} are independently selected from the group consisting of hydrogen and C₁₋₄alkyl;
each X is independently selected from F, Cl, Br and I;
m is selected from 0, 1, 2 and 3; and
n is selected from 0, 1, 2 and 3.

### Preferences of R¹, R², R^{O1}, R^{N1}, R^{N2} and X

In formula (I) above, none, one or more of R¹ may be present and none, one or more of R² may be present, depending on the values of m and n, respectively. The following preferences apply equally and independently to each of R¹ and R², if present.

Preferably R¹ is —OR^{O1} and R^{O1} is selected from the group consisting of hydrogen and methyl.

Preferably R² is —OR^{O1} and R^{O1} is selected from the group consisting of hydrogen and methyl.

Preferably R¹ is methyl. Preferably R² is methyl.

Preferably R¹ is —NR^{N1}R^{N2} and each R^{N1} and each R^{N2} are independently selected from hydrogen and methyl.

Preferably R² is —NR^{N1}R^{N2} and each R^{N1} and each R^{N2} are independently selected from hydrogen and methyl.

Preferably R¹ is —X and X is F. Preferably R² is —X and X is F.

Preferably R¹ is —CX₃ and each X is F. Preferably R¹ is —CX₃ and each X is F.

Preferably R^{O1} is selected from the group consisting of hydrogen and methyl.

Preferably R^{N1} and R^{N2} are independently selected from hydrogen and methyl.

Preferably X is F.

Preferably R¹ and R² are independently selected from the group selected from —Me, —OH, —OMe, —NH₂, —NHMe, —N(Me)₂, —F and —CF₃

### Preferences of m and n

Preferably the compound of the invention is phenytoin (m is 0 and n is 0):

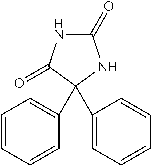 [SMILES: O=C1NC(=O)C(c2ccccc2)(c2ccccc2)N1]

In other embodiments, the compounds of the invention are phenytoin analogs, and one or both of m and n are greater than zero.

For convenience, the phenyl ring substituted with none, one or more R¹ groups shall be referred to as the first phenyl ring and the phenyl ring substituted with none, one or more R² groups shall be referred to as the second phenyl ring. It will be appreciated that the labeling of these rings is arbitrary because both phenyl rings are bonded to the same quaternary carbon atom. Accordingly, preferred values of m and n are interchangeable when the values for m and n are different because the same R¹ and R² substitution pattern will be produced. It will be clear, that any preference of R¹ will equally apply to R² if the values of m and n are interchanged.

Preferably, m may be 1 and n may be 0. Particularly preferred compounds when m is 1 and n is 0 are:

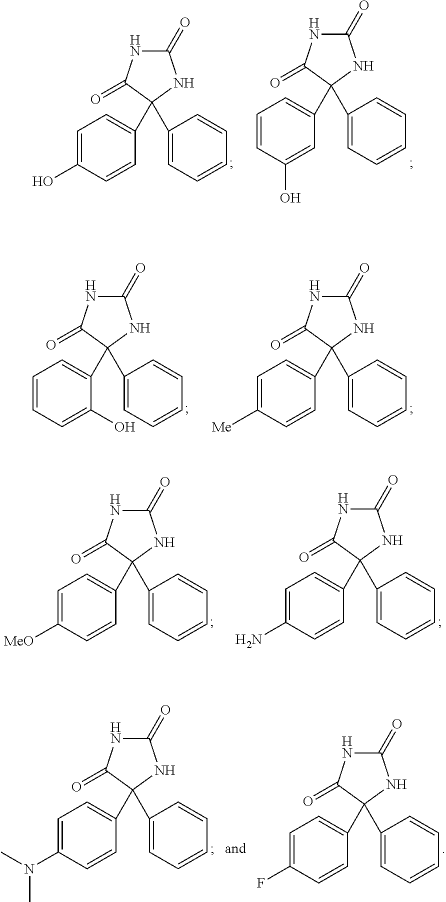

In further embodiments, m is 1 and n is 1. When m is 1 and n is 1, R¹ and R² may be the same group. It will be appreciated that when m is 1 and n is 1, R¹ and R² may be a different group.

Particularly preferred compounds when m is 1 and n is 1 are:

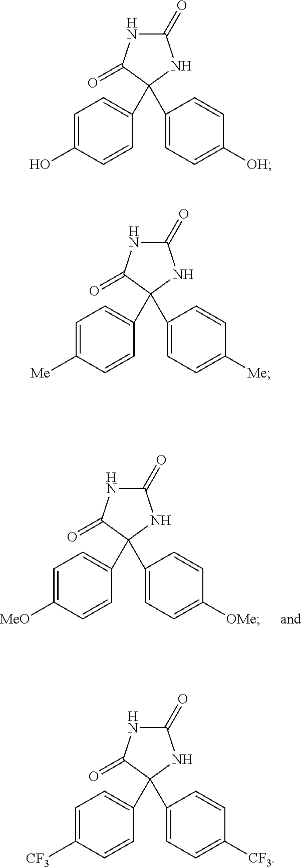 [SMILES: O=C1NC(=O)C(c2ccc(O)cc2)(c2ccc(O)cc2)N1 ; Cc1ccc(C2(c3ccc(C)cc3)NC(=O)NC2=O)cc1 ; COc1ccc(C2(c3ccc(OC)cc3)NC(=O)NC2=O)cc1 ; "and" ; O=C1NC(=O)C(c2ccc(C(F)(F)F)cc2)(c2ccc(C(F)(F)F)cc2)N1]

In further embodiments, m is 2 and n is 0. When m is 2 and n is 0, each R¹ may be the same group or a different group. Particularly preferred compounds when m is 2 and n is 0 are:

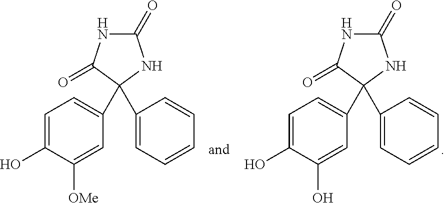 [SMILES: COc1cc(C2(c3ccccc3)NC(=O)NC2=O)ccc1O ; O=C1NC(=O)C(c2ccccc2)(c2ccc(O)c(O)c2)N1]

When m is 1, preferably the first phenyl ring is substituted with R¹ in the 4-position. When n is 1, preferably the second phenyl ring is substituted with R² in the 4-position.

When m is 2, preferably the first phenyl ring is substituted with one R¹ group in the 3-position and one R¹ group in the 4-position. When n is 2, preferably the second phenyl ring is substituted with one R² group in the 3-position and one R² group in the 4-position.

When m is 1 and n is 1, preferably the first phenyl ring is substituted with R¹ in the 4-position and the second phenyl ring is substituted with R² in the 4-position.

### Definitions and Further Preferences

### Alkyl:

As used herein the term “C₁₋₄alkyl”, unless otherwise specified, refers to a monovalent moiety obtained by removing a hydrogen atom from a hydrocarbon compound having from 1 to 4 carbon atoms, which may be aliphatic or alicyclic, or a combination thereof, which may be linear or branched, and which may be saturated, partially unsaturated, or fully unsaturated.

Examples of saturated linear C₁₋₄ alkyl groups include methyl, ethyl, n-propyl, and n-butyl.

An example of a saturated branched C₁₋₄ alkyl group is iso-propyl.

Examples of saturated alicyclic C₁₋₄ alkyl groups (which may also be referred to as “C₃₋₄ cycloalkyl” groups) include, but are not limited to, groups such as cyclopropyl and cyclobutyl, as well as substituted groups (e.g., groups which comprise such groups), such as methylcyclopropyl.

Unsaturated alkyl groups contain one or more double or triple bonds i.e. one or more carbon-carbon π bonds. Examples of unsaturated C₁₋₄ alkyl groups which have one or more carbon-carbon double bonds (also referred to as “C₂₋₄alkenyl” groups) include, but are not limited to, ethenyl (vinyl, —CH═CH₂), 2-propenyl (allyl, —CH—CH═CH₂), isopropenyl (—C(CH₃)═CH₂), and butenyl.

Examples of unsaturated C₁₋₄ alkyl groups which have one or more carbon-carbon triple bonds (also referred to as “C₂₋₄ alkynyl” groups) include, but are not limited to, ethynyl (ethinyl) and 2-propynyl (propargyl).

Examples of unsaturated alicyclic (carbocyclic) C₁₋₄ alkyl groups which have one or more carbon-carbon double bonds (also referred to as “C₃₋₄cycloalkenyl” groups) include, but are not limited to, unsubstituted groups such as cyclopropenyl and cyclobutenyl, as well as substituted groups (e.g., groups which comprise such groups) such as cyclopropenylmethyl.

### Isomers, Salts, Solvates, and Protected Forms

Certain compounds may exist in one or more particular geometric, optical, enantiomeric, diasteriomeric, epimeric, stereoisomeric, tautomeric, conformational, or anomeric forms, including but not limited to, cis- and trans-forms; E- and Z-forms; c-, t-, and r-forms; endo- and exo-forms; R-, S-, and meso-forms; D- and L-forms; d- and l-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and halfchair-forms; and combinations thereof, hereinafter collectively referred to as “isomers” (or “isomeric forms”).

Note that, except as discussed below for tautomeric forms, specifically excluded from the term “isomers”, as used herein, are structural (or constitutional) isomers (i.e. isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, metachlorophenyl. However, a reference to a class of structures may well include structurally isomeric forms falling within that class (e.g., C₁₋₇ alkyl includes n-propyl and iso-propyl; butyl includes n-, iso-, sec-, and tert-butyl; methoxyphenyl includes ortho-, meta-, and para-methoxyphenyl).

The above exclusion does not pertain to tautomeric forms, for example, keto-, enol-, and enolate-forms, as in, for example, the following tautomeric pairs: keto/enol (illustrated below), imine/enamine, amide/imino alcohol, amidine/amidine, nitroso/oxime, thioketone/enethiol, N-nitroso/hyroxyazo, and nitro/aci-nitro.

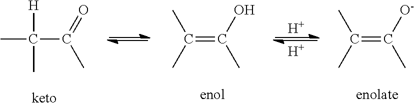

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C, O may be in any isotopic form, including ¹⁶O and ¹⁸O; and the like.

Unless otherwise specified, a reference to a particular compound includes all such isomeric forms, including (wholly or partially) racemic and other mixtures thereof Methods for the preparation (e.g. asymmetric synthesis) and separation (e.g., fractional crystallization and chromatographic means) of such isomeric forms are either known in the art or are readily obtained by adapting the methods taught herein, or known methods, in a known manner.

Unless otherwise specified, a reference to a particular compound also includes ionic, salt, solvate, and protected forms of thereof, for example, as discussed below.

It may be convenient or desirable to prepare, purify, and/or handle a corresponding salt of the active compound, for example, a pharmaceutically-acceptable salt. Examples of pharmaceutically acceptable salts are discussed in Berge, et al., J. Pharm. Sci., 66, 1-19 (1977).

For example, if the compound is anionic, or has a functional group which may be anionic (e.g., —COOH may be —COO⁻), then a salt may be formed with a suitable cation. Examples of suitable inorganic cations include, but are not limited to, alkali metal ions such as Na⁺ and alkaline earth cations such as C²⁺ and Mg²⁺, and other cations such as Al³⁺. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

If the compound is cationic, or has a functional group which may be cationic (e.g., —NH₂ may be —NH₃⁺), then a salt may be formed with a suitable anion. Examples of suitable inorganic anions include, but are not limited to, those derived from the following inorganic acids: hydrochloric, hydrobromic, hydroiodic, sulphuric, sulphurous, nitric, nitrous, phosphoric, and phosphorous. Examples of suitable organic anions include, but are not limited to, those derived from the following organic acids: acetic, propionic, succinic, glycolic, stearic, palmitic, lactic, malic, pamoic, tartaric, citric, gluconic, ascorbic, maleic, hydroxymaleic, phenylacetic, glutamic, aspartic, benzoic, cinnamic, pyruvic, salicyclic, sulfanilic, 2-acetyoxybenzoic, fumaric, phenylsulfonic, toluenesulfonic, methanesulfonic, ethanesulfonic, ethane disulfonic, oxalic, pantothenic, isethionic, valeric, lactobionic, and gluconic. Examples of suitable polymeric anions include, but are not limited to, those derived from the following polymeric acids: tannic acid, carboxymethyl cellulose.

In particular, the imidazolidine-dione ring of phenytoin and phenytoin analogs may form a salt with a suitable cation. Preferably, the compound of the invention is a sodium salt. A particularly preferred compound of the invention is the phenytoin sodium salt:

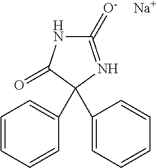

It may be convenient or desirable to prepare, purify, and/or handle a corresponding solvate of the active compound. The term “solvate” is used herein in the conventional sense to refer to a complex of solute (e.g. active compound, salt of active compound) and solvent. If the solvent is water, the solvate may be conveniently referred to as a hydrate, for example, a mono-hydrate, a di-hydrate, a tri-hydrate, etc.

It may be convenient or desirable to prepare, purify, and/or handle the active compound in a chemically protected form. The term “chemically protected form”, as used herein, pertains to a compound in which one or more reactive functional groups are protected from undesirable chemical reactions, that is, are in the form of a protected or protecting group (also known as a masked or masking group or a blocked or blocking group). By protecting a reactive functional group, reactions involving other unprotected reactive functional groups can be performed, without affecting the protected group; the protecting group may be removed, usually in a subsequent step, without substantially affecting the remainder of the molecule. See, for example, Protective Groups in Organic Synthesis (T. Green and P. Wuts, Wiley, 1999).

For example, a hydroxy group may be protected as an ether (—OR) or an ester (—OC(═O)R), for example, as: a t-butyl ether; a benzyl, benzhydryl (diphenylmethyl), or trityl (triphenylmethyl) ether; a trimethylsilyl or t-butyldimethylsilyl ether; or an acetyl ester (—OC(═O)CH₃, —OAc).

For example, an aldehyde or ketone group may be protected as an acetal or ketal, respectively, in which the carbonyl group (>C═O) is converted to a diether (>C(OR)₂), by reaction with, for example, a primary alcohol. The aldehyde or ketone group is readily regenerated by hydrolysis using a large excess of water in the presence of acid.

For example, an amine group may be protected, for example, as an amide or a urethane, for example, as: a methyl amide (—NHCO—CH₃); a benzyloxy amide (—NHCO—OCH₂C₆H₅, —NH—Cbz); as a t-butoxy amide (—NHCO—OC(CH₃)₃, —NH-Boc); a 2-biphenyl-2-propoxy amide (—NHCO—OC(CH₃)₂C₆H₄C₆H₅, —NH-Bpoc), as a 9-fluorenylmethoxy amide (—NH-Fmoc), as a 6-nitroveratryloxy amide (—NH-Nvoc), as a 2-trimethylsilylethyloxy amide (—NH-Teoc), as a 2,2,2-trichloroethyloxy amide (—NH-Troc), as an allyloxy amide (—NH-Alloc), as a 2(-phenylsulphonyl)ethyloxy amide (—NH-Psec); or, in suitable cases, as an N-oxide (>NO•).

For example, a carboxylic acid group may be protected as an ester for example, as: an C₁₋₇ alkyl ester (e.g. a methyl ester; a t-butyl ester); a C₁₋₇ haloalkyl ester (e.g., a C₁₋₇ trihaloalkyl ester); a triC₁₋₇ alkylsilyl-C₁₋₇ alkyl ester; or a C₅₋₂₀ aryl-C₁₋₇ alkyl ester (e.g. a benzyl ester; a nitrobenzyl ester); or as an amide, for example, as a methyl amide.

For example, a thiol group may be protected as a thioether (—SR), for example, as: a benzyl thioether; an acetamidomethyl ether (—S—CH₂NHC(═O)CH₃).

### Prodrugs

It is contemplated that some of the active compounds of the invention act in the form of prodrugs. In other words, the compounds are metabolized in the body to the active form.

In particular, prodrugs of phenytoin are known in the art. For example, see Ogiso et al., Biol. Pharm. Bull., 1993, 16(10), 1025-30 and Varia et al., J. Pharm. Sci., 1984, 73, 1069-1087, both of which are incorporated herein in their entirety.

Many of the prodrugs of phenytoin are substituted in one or both of the nitrogen atoms of the imidazolidine-dione ring. The nitrogen atoms may be substituted with any known N-atom prodrug substituent, including, but not limited to, alkyl, ether and ester groups.

A particularly preferred prodrug of phenytoin is the compound known as Phosphenytoin (or Fosphenytoin):

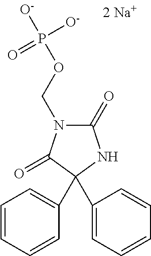 [SMILES: O=C1NC(c2ccccc2)(c2ccccc2)C(=O)N1COP(=O)([O-])[O-]]

In addition to phenytoin, preferred compounds of the invention are listed in the Table below:

| Substance | CAS Registry & name | Source |
|---|---|---|
| fosphenytoin | 93390-81-9 | J Pharm Sci |
| | 5,5-diphenyl-3- | 1984; 73(8): 1080 |
| | ((phosphonooxy)methyl)-2,4- | J Pharm Sci |
| | imidazolidinedione,, (SP-4-2) | 1984; 73(8): 1087 |
| hydroxyphenytoin | 2784-27-2 | Biomed Mass |
| | 5-(4-hydroxyphenyl)-5-phenyl-2,4- | Spectrom |
| | imidazolidinedione | 4(1): 36; 1977 |
| | | Clin Chem |
| | | 22(10): 1672; 1976 |
| | | Clin Chim Acta |
| | | 62: 73; 1975 |
| | | Clin Pharmacol Anti- |
| | | Epileptic Drugs W3 |
| | | WO925: 131; 1974c |
| | | J Chromatogr |
| | | 116(1): 53; 1976 |
| | | J Clin Invest |
| | | 51(5): 1164; 1972 |
| | | Proc Soc Exp Biol |
| | | Med 149: 371; 1975 |
| | | Res Commun Chem |
| | | Pathol Pharmacol |
| | | 19(2): 257; 1978 |
| 5-(3-hydroxyphenyl)-5- | 30074-03-4 | Drug Metab Dispos |
| phenylhydantoin | | 1980; 8(5): 295 |
| 5-phenyl-5-(4- | 53819-79-7 | Toxicol Appl |
| hydroxyphenyl)hydantoin | | Pharmacol |
| glucuronide | | 1981; 58(3): 510 |
| ropitoin hydrochloride | 56079-80-2 | Arzneim Forsch |
| | 2,4-Imidazolidinedione, 5-(4- | 1980; 30(1): 12 |
| | methoxyphenyl)-5-phenyl-3-(3-(4- | |
| | phenyl-1-piperidinyl)propyl)-, | |
| | monohydrochloride | |
| 5-(3,4-dihydroxy-1,5- | 28129-90-0 | Drug Metab Dispos |
| cyclohexadien-1-yl)-5- | | 1980; 8(5): 295 |
| phenylhydantoin | | |
| N-aminodiphenylhydantoin | 1224-08-4 | Pharmazie |
| | 2,4-Imidazolidinedione, 3-amino-5,5- | 1981; 36(11): 780 |
| | diphenyl | |
| 5-(3,4-dihydroxyphenyl)-5- | 35531-90-9 | J Pharmacol Exp Ther |
| phenylhydantoin | | 1983; 225(3): 630 |
| PC-796 | 21413-28-5 | |
| 5-p-methylphenyl-5- | 51169-17-6 | Clin Pharmacol Anti- |
| phenylhydantoin | | Epileptic Drugs |
| | | W3Wo925: 124; 1974c |
| 5-(2-hydroxyphenyl)-5- | 60919-11-1 | J Pharm Belg |
| phenylhydantoin | | 1982; 37(1): 47 |
| 1-acetyl-3-acetoxy-5′,5- | 56775-94-1 | Res Commun Chem |
| diphenylhydantoin | | Pathol Pharmacol |
| | | 1984; 44(2): 251 |
| 3-hydroxymethylphenytoin | 71919-15-8 | J Pharm Sci |
| N,N-dimethylglycine ester | Glycine, N,N-dimethyl-, (R) | 1984; 73(8): 1080 |
| | | J Pharm Sci |
| | | 1984; 73(8): 1087 |
| 3-(hydroxymethyl) | 92135-00-7 | J Pharm Sci |
| phenytoin N,N- | Carbonic acid, 5,5-diphenyl-3- | 1984; 73(8): 1080 |
| dimethylaminoethyl | ((phosphonooxy)methyl)-, (SP-4-2) | J Pharm Sci |
| carbonate | | 1984; 73(8): 1087 |
| 5-(4-hydroxy-3- | 36653-52-8 | Epilepsia |
| methoxyphenyl)-5- | | 1988; 29(6): 753 |
| phenylhydantoin | | |
| 3-pentanoyl-5,5- | 22506-76-9 | Biol Pharm Bull 1993 |
| diphenylhydantoin | | Oct; 16(10): 1025-30 |
| 3-(2-propylpentanoyl)-5,5- | 153735-26-3 | Biol Pharm Bull 1993 |
| diphenylhydantoin | | Oct; 16(10): 1025-30 |
| 5,5-bis(4- | 60348-77-8 | Drug Metab Disp |
| hydroxyphenyl)hydantoin | | 4(4): 349; 1976 |
| 3- | 21616-46-6 | J Pharm Sci |
| (hydroxymethyl)phenytoin | 2,4-Imidazolidinedione, 3- | 1984; 73(8): 1074 |
| | (hydroxymethyl)-5,5-diphenyl-, (7R- | |
| | (7alpha,8beta,10beta)) | |
| phenytoin dihydrodiol | 0 | J Chromatogr |
| | | 1985; 342(2): 323 |
| 4-aminophenytoin | 88168-80-3 | Int J |
| | | Immunopharmacol |
| | | 1987; 9(3): 391 |
| N,N-dichlorophenytoin | 100965-46-6 | Chem Res Toxicol |
| | | 1988 May- |
| | | Jun; 1(3): 148-51 |
| diphenylthiohydantoin | 21083-47-6 | |
| diphenylhydantoin-3- | 155707-42-9 | Res Commun Chem |
| phenyltricarbonylchromium | | Pathol Pharmacol |
| ethyl acetate | | 1994 Apr; 84(1): 81-92 |
| 5,5-diphenylhydantoin-3- | 0 | Res Commun Chem |
| valerate-bovine serum | | Pathol Pharmacol |
| albumin | | 1994 Apr; 84(1): 81-92 |
| phenytoin-1- | 0 | Biol Pharm Bull 1994 |
| methylnicotininate | | Oct; 17(10): 1425-9 |
| 2-cyanoguanidino- | 0 | J Pharm Sci 1996 |
| phenytoin | 2-(cyanoimino)-5,5-diphenyl-4- | Oct; 85(10): 1077-81 |
| | imidazolidinone | |
| phenytoin-bis- | 0 | Pharm Res 1997 |
| hydroxyisobutyrate | | Feb; 14(2): 251-3 |
| N-acetylphenytoin | 0 | Biol Pharm Bull |
| | | 1998; 21(10): 1084-9 |
| diphenylhydantoic acid | 0 | Regul Toxicol |
| | | Pharmacol 1999 |
| | | Feb; 29(1): 1-14 |
| N′-3-oxymethylglucuronide | 0 | Clin Chem 2001 |
| phenytoin | | May; 47(5): 910-8 |
| diphenylhydantil | 18749-95-6 | Arch Pharm |
| | (4,4′-Biimidazolidine)-2,2′,5,5′-tetrone, | 309(12): 1016; 1976 |
| | 4,4′-diphenyl | |
| 5-(4′-fluorophenyl)-5- | 0 | J Med Chem |
| phenylhydantoin | | 22(9): 1140; 1979 |
| Dantrolene | 7261-97-4 | Anaesthesia (2004) 59 |
| | 1-{[5-(4-nitrophenyl)-2- | (4): 364-73 |
| | furyl]methylideneamino}imidazolidine- | |
| | 2,4-dione | |
| Azumolene | 1-{[5-(4-bromophenyl)-2- | Dictionary of |
| | furyl]methylideneamino}imidazolidine- | Pharmacological |
| | 2,4-dione | Agents, vol. 1, p. 211 |

### Treatment & Prophylaxis

The compounds in the present invention are given to treat acute optic neuritis. Likewise the compounds in the present invention may be given prophylactically in respect of the loss of visual acuity which may otherwise follow onset of the acute optic neuritis.

The term “treatment,” as used herein in the context of treating a condition, pertains generally to treatment and therapy of a human, in which some desired therapeutic effect is achieved, for example, the inhibition of the progress of the condition, and includes a reduction in the rate of progress, a halt in the rate of progress, regression of the condition, amelioration of the condition, and cure of the condition. Treatment as a prophylactic measure (i.e., prophylaxis, prevention) is also included.

The term “therapeutically-effective amount,” as used herein, pertains to that amount of a compound of the invention, or a material, composition or dosage from comprising said compound, which is effective for producing some desired therapeutic effect, commensurate with a reasonable benefit/risk ratio, when administered in accordance with a desired treatment regimen.

Similarly, the term “prophylactically effective amount,” as used herein pertains to that amount of a compound of the invention, or a material, composition or dosage from comprising said compound, which is effective for producing some desired prophylactic effect, commensurate with a reasonable benefit/risk ratio, when administered in accordance with a desired treatment regimen.

“Prophylaxis” in the context of the present specification should not be understood to circumscribe complete success i.e. complete protection or complete prevention. Rather prophylaxis in the present context refers to a measure which is administered in advance of detection of a symptomatic condition with the aim of preserving health by helping to delay, mitigate or avoid that particular condition.

The term “treatment” includes combination treatments and therapies, in which two or more treatments or therapies are combined, for example, sequentially or simultaneously.

For example, as described above, it may be beneficial to combine treatment with a compound as described herein with one or more other (e.g., 1, 2, 3, 4) agents or therapies.

The particular combination would be at the discretion of the physician who would select dosages using his/her common general knowledge and dosing regimens known to a skilled practitioner.

The agents (i.e., a compound as described herein, plus one or more other agents) may be administered simultaneously or sequentially, and may be administered in individually varying dose schedules and via different routes. For example, when administered sequentially, the agents can be administered at closely spaced intervals (e.g., over a period of 5-10 minutes) or at longer intervals (e.g., 1, 2, 3, 4 or more hours apart, or even longer periods apart where required), the precise dosage regimen being commensurate with the properties of the therapeutic agent(s).

The agents (i.e., a compound as described here, plus one or more other agents) may be formulated together in a single dosage form, or alternatively, the individual agents may be formulated separately and presented together in the form of a kit, optionally with instructions for their use.

### Compositions/Formulations

While it is possible for the compound of the invention to be used (e.g., administered) alone, it is often preferable to present it as a composition or formulation.

Another aspect of the invention therefore provides a composition comprising a compound as described herein, and a pharmaceutically acceptable carrier or diluent.

In some embodiments, the composition is a pharmaceutical composition (e.g., formulation, preparation, medicament) comprising a compound as described herein, and a pharmaceutically acceptable carrier, diluent, or excipient.

In some embodiments, the composition is a pharmaceutical composition comprising at least one compound, as described herein, together with one or more other pharmaceutically acceptable ingredients well known to those skilled in the art, including, but not limited to, pharmaceutically acceptable carriers, diluents, excipients, adjuvants, fillers, buffers, preservatives, anti-oxidants, lubricants, stabilizers, solubilizers, surfactants (e.g., wetting agents), masking agents, coloring agents, flavoring agents, and sweetening agents.

In some embodiments, the composition further comprises other active agents, for example, other therapeutic or prophylactic agents.

Suitable carriers, diluents, excipients, etc. can be found in standard pharmaceutical texts. See, for example, Handbook of Pharmaceutical Additives, 2nd Edition (eds. M. Ash and I. Ash), 2001 (Synapse Information Resources, Inc., Endicott, New York, USA), Remington's Pharmaceutical Sciences, 20th edition, pub. Lippincott, Williams & Wilkins, 2000; and Handbook of Pharmaceutical Excipients, 2nd edition, 1994.

The term “pharmaceutically acceptable,” as used herein, pertains to compounds, ingredients, materials, compositions, dosage forms, etc., which are, within the scope of sound medical judgment, suitable for use in contact with the tissues of the subject in question (e.g., human) without excessive toxicity, irritation, allergic response, or other problem or complication, commensurate with a reasonable benefit/risk ratio. Each carrier, diluent, excipient, etc. must also be “acceptable” in the sense of being compatible with the other ingredients of the formulation.

The formulations may be prepared by any methods well known in the art of pharmacy. Such methods include the step of bringing into association the compound with a carrier which constitutes one or more accessory ingredients. In general, the formulations are prepared by uniformly and intimately bringing into association the compound with carriers (e.g., liquid carriers, finely divided solid carrier, etc.), and then shaping the product, if necessary.

The formulation may be prepared to provide for rapid or slow release; immediate, delayed, timed, or sustained release; or a combination thereof.

Formulations suitable for parenteral administration (e.g., by injection), include aqueous or non-aqueous, isotonic, pyrogen-free, sterile liquids (e.g., solutions, suspensions), in which the compound is dissolved, suspended, or otherwise provided (e.g., in a liposome or other microparticulate). Such liquids may additional contain other pharmaceutically acceptable ingredients, such as anti-oxidants, buffers, preservatives, stabilizers, bacteriostats, suspending agents, thickening agents, and solutes which render the formulation isotonic with the blood (or other relevant bodily fluid) of the intended recipient. Examples of excipients include, for example, water, alcohols, polyols, glycerol, vegetable oils, and the like. Examples of suitable isotonic carriers for use in such formulations include Sodium Chloride Injection, Ringer's Solution, or Lactated Ringer's Injection. Typically, the concentration of the compound in the liquid is from about 1 ng/ml to about 10 μg/ml, for example from about 10 ng/ml to about 1 μg/ml. The formulations may be presented in unit-dose or multi-dose sealed containers, for example, ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules, and tablets.

### Examples of Some Preferred Formulations

One aspect of the present invention pertains to a dosage unit (e.g., a pharmaceutical tablet or capsule) comprising 20 to 300 mg of a compound as described herein and optionally a pharmaceutically acceptable carrier, diluent, or excipient.

In some embodiments, the dosage unit is a tablet.

In some embodiments, the dosage unit is a capsule.

In some embodiments, the amount is 30 to 200 mg.

In some embodiments, the amount is about 30 mg.

In some embodiments, the amount is about 60 mg.

In some embodiments, the amount is about 100 mg.

In some embodiments, the amount is about 150 mg.

In some embodiments, the amount is about 200 mg.

In some embodiments, the pharmaceutically acceptable carrier, diluent, or excipient is or comprises one or both of a glyceride (e.g., Gelucire 44/14®; lauroyl macrogol-32 glycerides PhEur, USP) and colloidal silicon dioxide (e.g., 2% Aerosil 200®; Colliodal Silicon Dioxide PhEur, USP).

### Imaging of the Retinal Nerve Fiber Layer (RNFL) Using Optical Coherence Tomography (OCT)

It may be desired to monitor therapeutic efficacy (inhibition of axonal degeneration) directly or indirectly (via a surrogate biomarker).

Axonal loss cannot be quantified directly in vivo but RNFL atrophy correlates well with measures of visual function and can be measured sensitively and reproducibly using OCT.

More specifically, since the RNFL is composed only of unmyelinated axons, measuring its thickness represents a viable method of monitoring axonal loss. OCT has demonstrated RNFL and ganglion cell layer (GCL) thinning in MS⁶²⁻⁶⁴ and optic neuritis, and its use as a surrogate biomarker is supported by its correlation with visual outcome after optic neuritis. OCT is a non-invasive, accurate technique.

Because the lag that occurs in the full development of atrophy in the optic nerve⁴³ and in the retina⁴⁴ OCT measurements to confirm benefit can be made prior to treatment and after treatment e.g. 6 months after commencing treatment.⁵³

In one protocol, cross-sectional imaging of internal tissue microstructure is performed by measuring the echo time delay of back-scattered infrared light from a low coherence light source. Axial resolutions of 2-3 μm have now been obtained with ultrahigh-resolution OCT. There is a predilection for thinning of the RNFL in its temporal quadrant, which contains papillomacular fibers that are particularly susceptible to insults associated with optic neuritis^{51,65}. The high reproducibility of OCT has been confirmed by a number of studies, as reviewed by Stein et al⁶⁶.

Retinal nerve fibre layer (RNFL) images may be acquired by taking three circular 3.4 mm scans, centred on the optic disc, the mean of which can be used to express RNFL thickness. The thicknesses of the quadrants of the RNFL can also be automatically calculated by the OCT device software, and macular thickness maps acquired by making six radial scans centred on the fovea, and constructing a map from these scans. The images can be given a signal strength by the Stratus OCT device, with a maximum of 10.

### Visual Acuity

To help with the interpretation of any treatment effects on OCT measurements, and to assess the safety of treatment, changes in logMAR visual acuity, contrast letter sensitivity using 1.25% Sloan charts, and color vision using the Farnsworth-Munsell 100 hue test may be used. Abnormalities commonly found on examination in previous optic neuritis treatment trials were a reduction in visual acuity (89.5%), impairment of contrast sensitivity (98.2%) and color vision defects (93.8%)⁶⁷. Contrast letter acuity tests (Sloan charts) and contrast sensitivity tests (Pelli-Robson charts) appear to have the greatest capacity to identify and quantify visual dysfunction⁶⁸.

A number of patents and publications are cited herein in order to more fully describe and disclose the invention and the state of the art to which the invention pertains. Each of these references is incorporated herein by reference in its entirety into the present disclosure, to the same extent as if each individual reference was specifically and individually indicated to be incorporated by reference.

Throughout this specification, including the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Thus, for example, reference to “a pharmaceutical carrier” includes mixtures of two or more such carriers, and the like.

Ranges are often expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

Any sub-titles herein are included for convenience only, and are not to be construed as limiting the disclosure in any way.

The disclosure of all references cited herein, inasmuch as it may be used by those skilled in the art to carry out the invention, is hereby specifically incorporated herein by cross-reference.

### LITERATURE CITED

1. Kapoor R et al (2010) Lamotrigine for neuroprotection in secondary progressive multiple sclerosis: a randomised, double-blind, placebo-controlled, parallel-group trial. The Lancet Neurology; 9: 681-88.

2. McDonald W I (1983) The significance of optic neuritis. Trans Ophthal Soc UK; 103: 230-46.

3. Beck R W et al (2004) Visual function more than 10 years after optic neuritis: experience of the optic neuritis treatment trial. Am J Ophthalmol; 137: 77-83.

4. Plant G T (2008) Optic neuritis and multiple sclerosis. Curr Opin Neurol; 21: 16-21.

5. Bowden A N et al (1974) A trial of corticotrophin gelatin injection in acute optic neuritis. J Neurol Neurosurg Psychiatry; 37: 869-73.

6. Beck R W et al (1992) A randomized, controlled trial of corticosteroids in the treatment of acute optic neuritis. New Engl J Med; 326: 581-88.

7. Rawson M D et al (1966) Treatment of acute retrobulbar neuritis with corticotrophin. Lancet; 2:1044-46.

8. Rawson M D, Liversedge L A (1969) Treatment of retrobulbar neuritis with corticotrophin. Lancet; 2: 222.

9. Spoor T C, Rockwell D L (1988) Treatment of optic neuritis with intravenous megadose corticosteroids: a consecutive series. Ophthalmology; 95: 131-34.

10. Gould E S et al (1977) Treatment of optic neuritis by retrobulbar injection of triamcinolone. Br Med J; 1: 1495-97.

11. Beck R W, Cleary P A. (1993) Optic neuritis treatment trial. One-year follow-up results. Arch Ophthalmol; 111: 773-75.

12. Kapoor R et al (1998) Effects of intravenous methylprednisolone on outcome in MRI based prognostic subgroups in acute optic neuritis. Neurology; 50, 230-37.

13. Youl B D et al (1991) The pathophysiology of acute optic neuritis. An association of gadolinium leakage with clinical and electrophysiological deficits. Brain; 114:2437-50.

14. Moreau T et al (1996) Transient increase in symptoms associated with cytokine release in patients with multiple sclerosis. Brain; 119:225-37.

15. Redford E J et al (1997) Nitric oxide donors reversibly block axonal conduction: demyelinated axons are especially susceptible. Brain; 120:2149-57.

16. Frohman E M et al (2005) Characterizing the mechanisms of progression in multiple sclerosis: Evidence and new hypotheses for future directions. Arch Neurol; 62: 1345-56.

17. Ferguson B et al (1997) Axonal damage in acute multiple sclerosis lesions. Brain; 120:393-99

18. Trapp B D et al (1998) Axonal transection in the lesions of multiple sclerosis. N Engl J Med 338, 278-85.

19. Kolappan M et al (2009) Assessing structure and function of the afferent visual pathway in multiple sclerosis and associated optic neuritis. J Neurol; 256: 305-19.

20. Bechtold D A, Smith K J (2005) Sodium-mediated axonal degeneration in inflammatory demyelinating disease. J Neurol Sci; 233: 27-35.

21. Stys P K (2005) General mechanism of axonal damage and its prevention. J Neurol Sci; 233: 3-13.

22. Garthwaite G et al (2002) Nitric oxide toxicity in CNS white matter: an in vitro study using rat optic nerve. Neuroscience; 109: 145-55.

23. Lo A C et al (2003) Phenytoin protects spinal cord axons and preserves axonal conduction and neurological function in a model of neuroinflammation in vivo. J Neurophysiol; 90: 3566-71.

24. Kapoor R et al (2003) Blockers of sodium and calcium entry protect axons from nitric oxidemediated degeneration. Ann Neurol; 53: 174-80.

25. Bechtold D A et al (2004) Axonal protection using flecainide in experimental autoimmune encephalomyelitis. Ann Neurol; 55: 607-16.

26. Bechtold D A et al (2006) Axonal protection achieved in a model of multiple sclerosis using lamotrigine. J Neurol; 253: 1542-51.

27. Craner M J et al (2005) Sodium channels contribute to microglia/macrophage activation and function in EAE and MS. Glia; 15: 220-29.

28. Kapoor R (2008) Sodium channel blockers and neuroprotection in multiple sclerosis using lamotrigine. J Neurol Sci; 274: 54-56.

29. Compston A, Coles A J (2002) Multiple sclerosis. Lancet; 359: 1221-31.

30. Goodin D S et al (2002) Disease modifying therapies in multiple sclerosis: report of the Therapeutics and Technology Assessment Subcommittee of the American Academy of Neurology and the MS Council for Clinical Practice Guidelines. Neurology; 58: 169-78.

31. Mehta L R et al (2009) Proof of concept studies for tissue-protective agents in multiple sclerosis. Multiple Sclerosis; 15: 542-46.

32. Lublin F D et al (2003) Effects of relapses on development of residual deficit in multiple sclerosis. Neurology; 61: 1528-1532.

33. Hirst C et al (2008) Contribution of relapses to disability in multiple sclerosis. J Neurol; 255: 280-87.

34. Leandri M (2003) Therapy of trigeminal neuralgia secondary to multiple sclerosis. Expert Rev Neurother; 3: 661-71.

35. Fern R et al (1993) Pharmacological protection of CNS white matter during anoxia: actions of phenytoin, carbamazepine and diazepam. J Pharmacol Exp Ther; 266: 1549-55.

36. Edmonds H L Jr et al (2001) Topiramate as a neuroprotectant in a rat model of global ischaemia-induced neurodegeneration. Life Sci; 69, 2265-77.

37. Hains B C et al (2004) Sodium channel blockade with phenytoin protects spinal cord axons, enhances axonal conduction, and improves functional motor recovery after contusion SCI. Exp Neurol; 188: 365-77.

38. McLean M J and MacDonald R I (1983) Multiple actions of phenytoin on mouse spinal cord neurons in cell culture. J Pharmacol Exp Ther; 227: 779-89.

39. Yaari Y et al (1986) Phenytoin: mechanism of its anticonvulsive action. Ann Neurol; 20: 171-84.

40. Moll C et al (1991) Increase of sodium channels in demyelinated lesions of multiple sclerosis. Brain Res; 556, 311-16.

41. Smith K J et al (2001) Electrically active axons degenerate when exposed to nitric oxide. Ann Neurol; 49: 470-76.

42. Katz D et al (1993) Correlation between MRI findings and lesion development in chronic active multiple sclerosis. Ann Neurol; 34: 661-69.

43. Hickman S J et al (2004) A serial MRI study following optic nerve mean area in acute optic neuritis. Brain; 127: 2478-2505.

44. Henderson A P D et al (2010) A serial study of retinal changes following optic neuritis with sample size estimates for acute neuroprotection trials. Brain, epub ahead of print.

45. Molyneux P D et al. (2000) The effect of interferon beta-1b treatment on MRI measures of cerebral atrophy in secondary progressive multiple sclerosis. European Study Group on Interferon beta-1b in secondary progressive multiple sclerosis. Brain; 123: 2256-63.

46. Miller D H et al (2002) Measurement of atrophy in multiple sclerosis: pathological basis, methodological aspects and clinical relevance. Brain; 125: 1676-95.

47. Schmierer K et al (2004) Magnetization transfer ratio and myelin in postmortem multiple sclerosis brain. Ann Neurol; 56: 407-15.

48. Anderson V M et al (2007) Detecting treatment effects on brain atrophy in relapsing remitting multiple sclerosis: Sample size estimates. J Neurol; 254: 1588-1594.

49. Altmann D R et al (2009) Sample sizes for brain atrophy outcomes in trials for secondary progressive multiple sclerosis. Neurology; 72: 595-601.

50. Hickman S et al (2003) Corticosteroids do not prevent optic nerve atrophy following optic neuritis. J Neurol Neurosurg Psychiatry; 74, 1139-41.

51. Henderson A P et al (2008) An investigation of the retinal nerve fibre layer in progressive multiple sclerosis using optical coherence tomography. Brain; 131: 277-87.

52. Trip S A et al (2005) Retinal nerve fiber layer axonal loss and visual function in optic neuritis. Ann Neurol; 58: 383-91.

53. Barkhof F et al (2009) Imaging outcomes for neuroprotection and repair in multiple sclerosis trials. Nat Rev Neurol; 5: 256-66.

54. Miller D H et al (2003) A controlled trial of natalizumab for relapsing multiple sclerosis. N Engl J Med; 348: 15-23.

55. Rodriguez M et al (1995) Optic neuritis: A population-based study in Olmsted County, Minnesota. Neurology; 45: 244-50.

56. MacDonald B K et al (2000) The incidence and lifetime prevalence of neurological disorders in a prospective community-based study in the UK. Brain; 123: 665-76.

57. Barnes D et al (1997) Randomized trial of oral and intravenous methylprednisolone in acute relapses of multiple sclerosis. Lancet; 349: 902-06.

58. Miller D M et al (2000) A meta-analysis of methylprednisolone in recovery from multiple sclerosis exacerbations, Mult Scler; 6: 262-67.

59. Black J A et al (2007) Exacerbation of experimental autoimmune encephalomyelitis after withdrawal of phenytoin and carbamazepine. Ann Neurol; 62: 21-33.

60. Noseworthy J H et al (1994) The impact of blinding on the results of a randomized, placebo controlled multiple sclerosis clinical trial. Neurology 44, 16-20.

61. Kallenbach K, Frederiksen J (2007) Optical coherence tomography in optic neuritis and multiple sclerosis: a review. Eur J Neurol; 14: 841-49.

62. Parisi V et al (1999) Correlation between morphological and functional retinal impairment in multiple sclerosis patients. Invest Ophthalmol Vis Sci; 40: 2520-27.

63. Fisher J B et al (2006) Relation of visual function to retinal nerve fiber layer thickness in multiple sclerosis. Ophthalmology; 113: 324-32.

64. Sepulcre Jet al (2007) Diagnostic accuracy of retinal abnormalities in predicting disease activity in MS. Neurology; 68: 1488-94.

65. Sergott R C et al (2007) The role of optical coherence tomography in multiple sclerosis: expert panel consensus. J Neurol Sci; 263: 3-14.

66. Stein D M et al (2004) Imaging in glaucoma. Ophthalmol Clin North Am; 17: 33-52.

67. Optic Neuritis Study Group (1991) The clinical profile of optic neuritis. Experience of the Optic Neuritis Treatment Trial. Arch Ophthalmol; 109: 1673-78.

68. Balcer L J et al (2003) Contrast letter acuity as a visual component for the Multiple Sclerosis Functional Composite. Neurology; 61: 1367-73.

69. Senn S J (1997) Statistical Issues in Drug Development. Wiley: Chichester.

70. Swadron S P et al (2004) A comparison of phenytoin-loading techniques in the emergency department. Acad Emerg Med; 11: 244-52.

71. Sakurai M et al (1992) Lidocaine unmasks silent demyelinative lesions in multiple sclerosis. Neurology 42, 2088-93.

72. Knowles S R et al (1999) Anticonvulsant hypersensitivity syndrome: incidence, prevention and management. Drug Safety; 21: 489-501.

73. Leppik I E et al (1985) Seasonal incidence of phenytoin allergy unrelated to plasma levels. Arch Neurol; 42: 120-22.

74. Craig S (2005) Phenytoin poisoning. Neurocrit Care; 3: 161-70.

1. A method of treating acute demyelinating optic neuritis in a patient suffering therefrom, which method comprises administering a compound to the patient, wherein the compound is phenytoin or an analog or prodrug thereof.

2. The method of claim 1 wherein the compound is a neuroprotectant and the treatment improves preservation of vision after onset of the acute demyelinating optic neuritis.

3. The method of claim 2 wherein neuroprotection is achieved by partial blocking of voltage-gated sodium channels in axonal and immune cell membranes thereby reducing the entry of sodium into the respective cell in vivo.

4. The method of claim 1 wherein the compound is selected from compounds of formula (I): 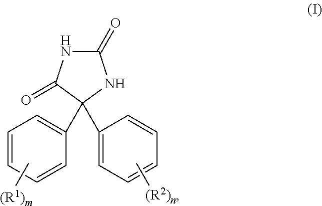
and their salts, solvates, hydrates, prodrugs and isomers thereof, wherein:
each R¹ and each R² is independently selected from the group consisting of C₁₋₄alkyl, —OR^{O1}, —NR^{N1}R^{N2}, —X, and —CX₃;
each R^{O1}, each R^{N1} and each R^{N2} is independently selected from the group consisting of hydrogen and C₁₋₄alkyl;
each X is independently selected from F, Cl, Br and I;
m is selected from 0, 1, 2 and 3; and
n is selected from 0, 1, 2 and 3.

5. The method of claim 4 wherein m is 0 and n is 0 and the compound is phenytoin: 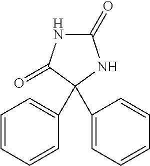 [SMILES: O=C1NC(=O)C(c2ccccc2)(c2ccccc2)N1]
or a salt thereof.

6. The method of claim 4 wherein the compound is an analog of phenytoin.

7. The method of claim 4 wherein the compound is a pro-drug of phenytoin.

8. The method of claim 1 wherein the compound is selected from the group consisting of: fosphenytoin, hydroxyphenytoin, 5-(3-hydroxyphenyl)-5-phenylhydantoin, 5-phenyl-5-(4-hydroxyphenyl)hydantoin glucuronide, ropitoin, ropitoin hydrochloride, 5-(2-hydroxyphenyl)-5-phenylhydantoin, 5-(3,4-dihydroxy-1,5-cyclohexadien-1-yl)-5-phenylhydantoin, N-aminodiphenylhydantoin, 5-(3,4-dihydroxyphenyl)-5-phenylhydantoin, PC-796, 5-p-methylphenyl-5-phenylhydantoin, 1-acetyl-3-acetoxy-5′,5-diphenylhydantoin, 3-hydroxymethylphenytoin N,N-dimethylglycine ester, 3-(hydroxymethyl)phenytoin N,N-dimethylaminoethyl carbonate, 5-(4-hydroxy-3-methoxyphenyl)-5-phenylhydantoin, 3-pentanoyl-5,5-diphenylhydantoin, 3-(2-propylpentanoyl)-5,5-diphenylhydantoin, 5,5-bis(4-hydroxyphenyl)hydantoin, 3-(hydroxymethyl)phenytoin, phenytoin dihydrodiol, 4-aminophenytoin, N,N-dichlorophenytoin, diphenylthiohydantoin, diphenylhydantoin-3-phenyltricarbonylchromium ethyl acetate, 5,5-diphenylhydantoin-3-valerate-bovine serum albumin, phenytoin-1-methylnicotininate, 2-cyanoguanidinophenytoin, phenytoin-bis-hydroxyisobutyrate, N-acetylphenytoin, diphenylhydantoic acid, N′-3-oxymethylglucuronide phenytoin, diphenylhydantil, 5-(4′-fluorophenyl)-5-phenylhydantoin, Dantrolene, Azumolene, 5,5-bis(4-trifluoromethylphenyl)hydantoin, 5,5-bis(4-methylphenyl)hydantoin, 5,5-bis(4-methoxyphenyl)hydantoin, 5-(4-methoxyphenyl)-5-phenylhydantoin, and 5-(4-dimethylaminophenyl)-5-phenylhydantoin.

9. The method of claim 1, wherein the method comprises administering to said patient a prophylactically or therapeutically effective amount of the compound in the form of a pharmaceutical composition.

10. The method of claim 1, wherein
(a) the compound is administered orally,
(b) the compound is administered such as to achieve a plasma concentration of 10-20 mg/ml, and/or
(c) the compound is administered at a dosage in the range of about 1 mg to about 25 mg per kilogram body weight of the subject per day.

11. The method of claim 1, wherein the compound is administered as loading then maintenance doses.

12. The method of claim 11 wherein
(a) the loading dose is between about 10 and 20 mg/kg/day,
(b) the maintenance dose is between 1 and 10 mg/kg/day,
(c) the maximum daily maintenance dose is less than 500, 400 or 300 mg/day,
(d) the loading dose is about 15 mg/kg, optionally rounded up to the nearest 100 mg, divided into three equal doses given once daily on 3 consecutive days; and/or
(e) the daily maintenance dose is about 4 mg/kg once daily, optionally rounded up to the nearest 50 mg, with a maximum of 300 mg.

13. The method of claim 9, wherein the treatment comprises administering the compound according to one of the following dosage regimes: about 50 mg, 3 times daily; about 100 mg, 3 times daily; about 150 mg, 2 times daily; and about 200 mg, 2 times daily.

14. The method of claim 1, wherein the patient has a diagnosis of MS.

15. The method of claim 1, wherein the patient does not have a diagnosis of MS.

16. The method of claim 1, wherein the patient has a visual acuity prior to treatment of less than 6/9 (M), 20/30 (Ft), or above LogMAR 0.20.

17. The method of claim 1, wherein
(a) the treatment takes place within 1, 2, 3, 4, 5, 6, 7, 8, 9, 10, 11, 12, 13 or 14 days of onset of optic neuritis, and wherein the treatment optionally takes place within 1, 2, or 3 days of onset of optic neuritis;
(b) the treatment is sustained until both inflammation and axonal membrane readaptation have subsided;
(c) the treatment takes place for about or at least 4, 5, 6, 7, 8, 9, 10, 11, 12, 13, 14, 15, 16, 17, 18, 19, 20 weeks, or about or at least 1, 2, 3, 4, 5, or 6 months from the onset of optic neuritis; and/or
(d) wherein the treatment takes place for less than 4, 5, or 6 months from the onset of optic neuritis and wherein the treatment is optionally for about 14 weeks or 3 months.

18. The method of claim 1, wherein the treatment is combined with other therapies which are symptomatic or disease modifying, wherein the treatment is optionally combined with treatment with an oral prednisone.

19. The method of claim 6, wherein m is not 0 or n is not 0.

20. A method as claimed in claim 19, wherein
(a) R¹ is —OR^{O1} and R^{O1} is selected from the group consisting of hydrogen and methyl; R² is —OR^{O1} and R^{O1} is selected from the group consisting of hydrogen and methyl;
(b) m is 1 and n is 0 and the compound is selected from: 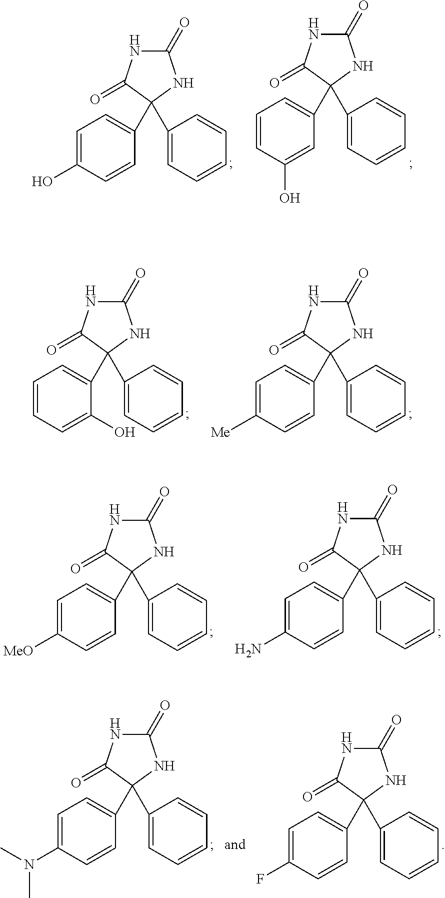
(c) m is 1 and n is 1 and the compound is selected from: 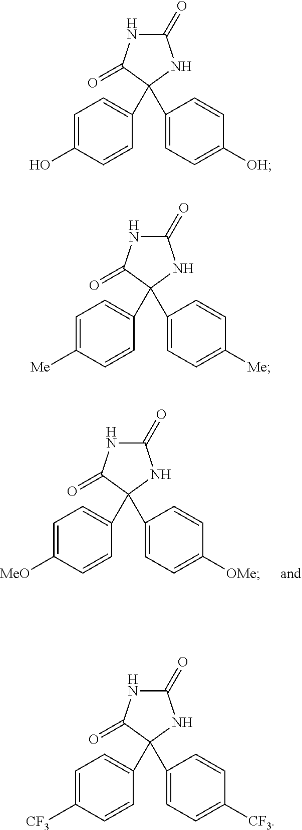
or
(d) m is 2 and n is 0 and the compound is selected from: 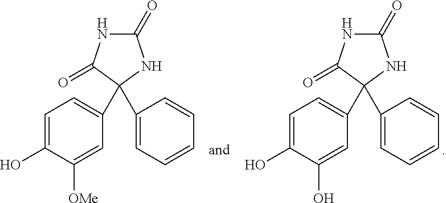 [SMILES: COc1cc(C2(c3ccccc3)NC(=O)NC2=O)ccc1O ; O=C1NC(=O)C(c2ccccc2)(c2ccc(O)c(O)c2)N1]
